SEQUENCE LISTING (14 sequences)

SEQ ID NO: 1 - DNA, 517 bp, Homo sapiens
  CDS (1)..(330)
ggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaagtatcagcccccatctaccaacaagaacacgaagtctcagagaaggaaaggaagtacatttgaagaacacaagtagagggagtgcaggaaacaagaactacaggatgtagaagacccttctgaggagtgaagaaggacaggccaccgcaggaccctttgctctgcacagttacctgtaaacattggaataccggccaaaaaataagtttgatcacatttcaaagatggcatttcccccaatgaaatacacaagtaaacat

SEQ ID NO: 2 - protein, 110 aa, Homo sapiens
GPETLCGAELVDALQFVCGDRGFYFNKPTGYGSSSRRAPQTGIVDECCFRSCDLRRLEMYCAPLKPAKSARSVRAQRHTDMPKTQKYQPPSTNKNTKSQRRKGSTFEEHK

SEQ ID NO: 3 - DNA, 539 bp, Rattus norvegicus
  CDS (1)..(333)
ggaccagagaccctttgcggggctgagctggtggacgctcttcagttcgtgtgtggaccaaggggcttttacttcaacaagcccacagtctatggctccagcattcggagggcaccacagacgggcattgtggatgagtgttgcttccggagctgtgatctgaggaggctggagatgtactgtgtccgctgcaagcctacaaagtcagctcgttccatccgggcccagcgccacactgacatgcccaagactcagaagtcccagcccctatcgacacacaagaaaaggaagctgcaaaggagaaggaaaggaagtacacttgaagaacacaagtagaggaagtgcaggaaacaagacctacagaatgtaggaggagcctcccgaggaacagaaaatgccacgtcaccgcaagatcctttgctgcttgagcaacctgcaaaacatcggaacacctgccaaatatcaataatgagttcaatatcatttcagagatgggcatttccctcaatgaaatacacaagtaaacattcccggaattc

SEQ ID NO: 4 - protein, 111 aa, Rattus norvegicus
GPETLCGAELVDALQFVCGPRGFYFNKPTVYGSSIRRAPQTGIVDECCFRSCDLRRLEMYCVRCKPTKSARSIRAQRHTDMPKTQKSQPLSTHKKRKLQRRRKGSTLEEHK

SEQ ID NO: 5 - DNA, 523 bp, Oryctolagus cuniculus
  CDS (1)..(333)
ggaccggagacgctctgcggtgctgagctggtggatgctcttcagttcgtgtgtggagacaggggcttttatttcaacaagcccacaggatacggctccagcagtcggagggcacctcagacaggcatcgtggatgagtgctgcttccggagctgtgatctgaggaggctggagatgtactgtgcacccctcaagccggcaaaggcagcccgctccgtccgtgcccagcgccacaccgacatgcccaagactcagaagtatcagcctccatctaccaacaagaaaatgaagtctcagaggagaaggaaaggaagtacatttgaagaacacaagtagagggagtgcaggaaacaagaactacaggatgtaggaagacccttctgaggagtgaagaaggacaggccaccgcaggaccctttgctctgcacagttacctgtaaacattggaataccggccaaaaaataagtttgatcacatttcaaagatggcatttcccccaatgaaatacacaagtaaacattc

SEQ ID NO: 6 - protein, 111 aa, Oryctolagus cuniculus
GPETLCGAELVDALQFVCGDRGFYFNKPTGYGSSSRRAPQTGIVDECCFRSCDLRRLEMYCAPLKPAKAARSVRAQRHTDMPKTQKYQPPSTNKKMKSQRRRKGSTFEEHK

SEQ ID NO: 7 - DNA, 10 bp, Artificial Sequence
  Description of Artificial Sequence translation initiation sequence
gccaccatgg

SEQ ID NO: 8 - DNA, 10 bp, Artificial Sequence
  Description of Artificial Sequence translation initiation sequence
gcccccatgg

SEQ ID NO: 9 - DNA, 317 bp, Homo sapiens
  CDS (1)..(315)
ggaccggagacgctctgcggggctgagctggtggatgctcttcagttcgtgtgtggagacaggggcttttatttcaacaagcccacagggtatggctccagcagtcggagggcgcctcagacaggcatcgtggatgagtgctgcttccggagctgtgatctaaggaggctggagatgtattgcgcacccctcaagcctgccaagtcagctcgctctgtccgtgcccagcgccacaccgacatgcccaagacccagaaggaagtacatttgaagaacgcaagtagagggagtgcaggaaacaagaactacaggatgag

SEQ ID NO: 10 - protein, 105 aa, Homo sapiens
GPETLCGAELVDALQFVCGDRGFYFNKPTGYGSSSRRAPQTGIVDECCFRSCDLRRLEMYCAPLKPAKSARSVRAQRHTDMPKTQKEVHLKNASRGSAGNKNYRM

SEQ ID NO: 11 - DNA, 487 bp, Rattus norvegicus
  CDS (1)..(315)
ggaccagagaccctttgcggggctgagctggtggacgctcttcagttcgtgtgtggaccaaggggcttttacttcaacaagcccacagtctatggctccagcattcggagggcaccacagacgggcattgtggatgagtgttgcttccggagctgtgatctgaggaggctggagatgtactgtgtccgctgcaagcctacaaagtcagctcgttccatccgggcccagcgccacactgacatgcccaagactcagaaggaagtacacttgaagaacacaagtagaggaagtgcaggaaacaagacctacagaatgtaggaggagcctcccgaggaacagaaaatgccacgtcaccgcaagatcctttgctgcttgagcaacctgcaaaacatcggaacacctgccaaatatcaataatgagttcaatatcatttcagagatgggcatttccctcaatgaaatacacaagtaaacattcccggaattc

SEQ ID NO: 12 - protein, 105 aa, Rattus norvegicus
GPETLCGAELVDALQFVCGPRGFYFNKPTVYGSSIRRAPQTGIVDECCFRSCDLRRLEMYCVRCKPTKSARSIRAQRHTDMPKTQKEVHLKNTSRGSAGNKTYRM

SEQ ID NO: 13 - DNA, 471 bp, Oryctolagus cuniculus
  CDS (1)..(315)
ggaccggagacgctctgcggtgctgagctggtggatgctcttcagttcgtgtgtggagacaggggcttttatttcaacaagcccacaggatacggctccagcagtcggagggcacctcagacaggcatcgtggatgagtgctgcttccggagctgtgatctgaggaggctggagatgtactgtgcacccctcaagccggcaaaggcagcccgctccgtccgtgcccagcgccacaccgacatgcccaagactcagaaggaagtacatttgaagaacacaagtagagggagtgcaggaaacaagaactacaggatgtaggaagacccttctgaggagtgaagaaggacaggccaccgcaggaccctttgctctgcacagttacctgtaaacattggaataccggccaaaaaataagtttgatcacatttcaaagatggcatttcccccaatgaaatacacaagtaaacattc

SEQ ID NO: 14 - protein, 105 aa, Oryctolagus cuniculus
GPETLCGAELVDALQFVCGDRGFYFNKPTGYGSSSRRAPQTGIVDECCFRSCDLRRLEMYCAPLKPAKAARSVRAQRHTDMPKTQKEVHLKNTSRGSAGNKNYRM

(19) United States
(12) Patent Application Publication
(10) Pub. No.: US 2006/0058239 A1
(43) Pub. Date: Mar. 16, 2006

(54) Use of the insulin-like-growth factor I isoform MGF for the treatment of neurological disorders

(76) Inventors: Ian Johnson, London (GB); Geoffrey Goldspink, London (GB)

Correspondence Address: NIXON & VANDERHYE, PC, 901 NORTH GLEBE ROAD, 11TH FLOOR, ARLINGTON, VA, 22203, US

(21) Appl. No.: 11/228,458

(22) Filed: Sep. 19, 2005

Related U.S. Application Data
(62) Division of application No. 10/130,211, filed on Sep. 23, 2002, now abandoned.

(30) Foreign Application Priority Data
Nov. 15, 1999 (GB) 9926968.0

(51) Int. Cl. A61K 48/00; A61K 38/30

(52) U.S. Cl. 514/12; 514/44

(57) ABSTRACT

The invention relates to the treatment of neurological disorders with the Insulin-like Growth Factor I (IGF-I) isoform known as mechano growth factor (MGF).

### FIELD OF THE INVENTION

The invention relates to the treatment of neurological disorders with the Insulin-like Growth Factor I (IGF-I) isoform known as mechano growth factor (MGF).

### BACKGROUND OF THE INVENTION

Mammalian IGF-I polypeptides have a number of isoforms, which arise as a result of alternative mRNA splicing. Broadly, there are two types of isoform, liver-type isoforms and non-liver ones. Liver-type isoforms may be expressed in the liver or elsewhere but, if expressed elsewhere, are equivalent to those expressed in the liver. They have a systemic action and are the main isoforms in mammals. Non-liver isoforms are less common and some are believed to have an autocrine/paracrine action. The latter type has been detected in skeletal and cardiac muscle but only following a mechanical overload.

The terminology for the IGF-I splice variants is based on the liver isoforms (Chew et al. 1995) and has not fully evolved to take into account those produced by non-liver tissues. The latter are controlled to some extent by a different promoter (promoter 1) to the liver IGF-I isoforms, which respond to hormones and are under the control of promoter 2 (Layall, 1996).

For the purposes of this invention, two isoforms are of particular interest. These are both expressed in skeletal muscle, though it has only recently been appreciated that two muscle isoforms exist. The first isoform is muscle liver-type IGF-I or L.IGF-I (systemic type), which is of interest mainly for comparative purposes. The second is mechano-growth factor or MGF (autocrine/paracrine type).

These are alternative splice variants. Exons 1 and 2 are alternative leader exons (Tobin et al, 1990; Jansen et al, 1991 ) with distinct transcription start sites which are differentially spliced to common exon 3. Exons 3 and 4 code for the mature IGF-I peptide (B, C, A and D domains) as well as the first 16 amino acid of the E domain. Exons 5 and 6 each encodes an alternative part of a distinct extension peptide, the E domain. This is followed by the termination codons of precursor IGF-I, 3′ untranslated regions and poly(A) addition signal sites (Rotwein el al. 1986). A further difference between the two isoforms is that MGF is not glycosylated and is therefore smaller. It has also been shown to be less stable. It may thus have a shorter half-life.

It has been shown that MGF, which is not detectable in skeletal muscle unless it is subjected to exercise or stretch (Yang et al, 1996), has exons 4, 5 and 6 whilst the muscle L.IGF-I has exons 4 and 6. Exon 5 in MGF has an insert of 52 bp which changes the 3′ reading frame and hence the carboxy end of the peptide. In addition, MGF has been detected in overloaded cardiac muscle (Skarli el al, 1998).

Functional epitope mapping of IGF-I using a battery of monoclonal antibodies (Ma\check{n}ies et al, 1997) has shown that the carboxy terminus (3′ end) of IGF-I is important in determining the affinity of the peptide for a particular receptor and/or binding protein.

MGF mRNA is not detected in dystrophic muscle even when it is subjected to stretch. The inability of muscle in both the autosomal- and dystrophin-deficient dystrophies to respond to overload by stretch (Goldspink et al, 1996) indicates that the cytoskeleton may be involved in the transduction mechanism. It is probable that there is a basic mechanism that detects muscle overload and which results in the expression of both variant forms of IGF.

Thus, MGF is known to be expressed in skeletal and cardiac muscle tissue in response to stretch and exercise and as a result is believed to be involved in repair of damage to muscle (Yang et al, 1996; WO97/33997). This has been confirmed more recently by McKoy et al (1999).

### SUMMARY OF THE INVENTION

The Inventors have now identified a new and surprising property of MGF.

Plasmids containing MGF DNA operably linked to expression signals capable of securing expression in muscles were prepared and injected intramuscularly into rats. Expression of MGF in vivo resulted. To investigate the effect of MGF on the animal's nerves, the right-facial nerve was damaged by avulsion in some animals and crushing in others. Similar experiments were performed with plasmids capable of expressing L.IGF-I and control experiments were also carried out using equivalent “empty” plasmids lacking an MGF or L.IGF-I coding sequence, and with non-operated rats.

The surgical procedures carried out normally result in massive motoneurone loss, and that was the case in the control animals. However, use of L.IGF-I reduced motoneurone loss to about 50% and use of MGF reduced motoneurone loss to about 20%. Although both isoforms were found to be effective in promoting motoneurone rescue, MGF was, surprisingly, more than twice as effective as L.IGF-I. This opens up the possibility of using MGF in the treatment of neurological disorders, especially motoneurone disorders. Additionally, it should be noted that this is the first time that altered availability of neurotrophic factors to intact adult motoneurones has been shown to affect a subsequent response to injury and also that this is the first time that intramuscular gene transfer using plasmid DNA has been shown to be an effective strategy for motoneuroneal rescue.

IGF-I isoforms have specific binding proteins which determine their action, particularly in terms of which tissues the isoform takes effect in. It appears that the binding protein for MGF is located in the central nervous system (CNS) as well as in skeletal and cardiac muscle. This may explain its greater effectiveness. Also, the fact that MGF is not glycosylated and thus smaller than L.IGF-I may facilitate its transfer from the muscle to the motor neuron cell bodies in the CNS.

These findings have general applicability to the treatment of neurological disorders and are surprising because MGF had previously only been detected in cardiac muscle and skeletal muscle under stretch/exercise. Chew (1995) suggests that an IGF-I Ec form is found in the liver. However, this is detectable in very low amounts and may be due to leaky transcription. Therefore, it had previously been believed that MGF was a muscle-specific isoform whereas it has now emerged that it is also implicated in repairing damage to the nervous system and can thus form the basis of treatments for disorders of the nervous system.

Accordingly, the invention provides:

Use of an MGF (mechano-growth factor) Insulin-like Growth Factor I (IGF-I) isoform comprising amino acid sequences encoded by nucleic acid sequences of IGF-I exons 4, 5 and 6 in the reading frame of MGF and having the ability to reduce motoneurone loss by 20% or greater in response to nerve avulsion, in the manufacture of a medicament for the treatment of a neurological disorder.

The invention also provides:

Use of a polynucleotide encoding an MGF IGF-I isoform of the invention in the manufacture of a medicament for the treatment of a neurological disorder.

The invention also provides:

A product comprising an MGF IGF-I isoform of the invention or an MGF-encoding polynucleotide of the invention and another neurologically active agent for simultaneous, separate or sequential use in the treatment of a neurological disorder.

The invention also provides:

A pharmaceutical composition comprising an MGF IGF-I isoform of the invention or an MGF-encoding polynucleotide of the invention, another neurologically active agent and a pharmaceutically acceptable carrier.

The invention also provides:

A method of treating a neurological disorder comprising administering to a subject in need of thereof an effective amount of an IGF-I isoform of the invention or a nucleic acid of the invention, optionally in combination with another neurologically active agent.

### BRIEF DESCRIPTION OF THE DRAWINGS

FIG. 1: Total numbers of motoneurones in the facial motor nucleus

KEY

| | |
|---|---|
| 1: | normal |
| 2: | 1 month crush |
| 3: | 1 month avulsion |
| 4: | plasmid only - 1 month avulsion |
| 5: | IGF-1 plasmid - 1 month avulsion |
| 6: | MGF plasmid - 1 month avulsion |

right: operated side;
left: non-operated side

FIG. 2: Avulsion (control experiments)

(a) Low magnification view of a transverse section through the brainstem at the level of the facial nucleus, I month following facial nerve avulsion. Numbers of motoneurones in the facial nucleus of the operated side (b) are markedly reduced compared to the non-operated nucleus (arrow and inset c). 70 μm vibratome section stained with YOYO and viewed using epifluorescence.

FIG. 3: Plasmid experiments

(a) Low magnification view of the brainstem at the level of the facial nucleus. Plasmid DNA without any gene insert was injected into the right snout muscle. 7 days later the right facial nerve was avulsed and the animal allowed to survive for 1 month. Like the effect of avulsion only (FIG. 1), numbers of motoneurones in the facial nucleus of the operated side (c) are markedly reduced compared to the non-operated nucleus (arrow and inset b). 70 μm vibratome section stained with YOYO and viewed using epiflourescence.

FIG. 4: MGF plasmid experiments

(a) Low magnification view of the brainstem at the level of the facial nucleus. Plasmid DNA containing the rat MGF gene was injected into the right snout muscle. 7 days later the right facial nerve was avulsed and the animal allowed to survive for 1 month. Numbers of motoneurones in the facial nucleus of the operated side (b) are similar to the non-operated nucleus (arrow and inset c). 70 μm vibratome section stained with YOYO and viewed using epiflourescence.

FIG. 5: cDNA and amino acid sequence of human MGF, showing its exon structure

FIG. 6: cDNA and amino acid sequence of rat MGF, showing its exon structure

FIG. 7: cDNA and amino acid sequence of rabbit MGF, showing its exon structure

FIG. 8: cDNA and amino acid sequence of human L.IGF-I, showing its exon structure

FIG. 9: cDNA and amino acid sequence of rat L-IGF-I, showing its exon structure

FIG. 10: cDNA and amino acid sequence of rabbit L-IGF-I, showing its exon structure

FIG. 11: Sequence alignment, illustrating exon structure of human, rat and rabbit MGF and L-IGF-I, and highlighting similarities and differences

### DETAILED DESCRIPTION OF THE INVENTION

The present invention concerns the use of MGF in the treatment of neurological disorders, preferably motoneurone disorders.

MGF Polypeptides and Polynucleotides

Polypeptides

MGF stands for mechano-growth factor (cf. McKoy et al 1999). As discussed above and explained in more detail in Chew et al (1995), Yang et al (1996) and McKoy et al (1999), MGF is an alternatively spliced variant of IGF-I. Liver-type IGF-I comprises amino acids encoded by exons 4 and 6 whereas MGF comprises amino acids encoded by exons 4, 5 and 6. MGF also has an altered reading frame at its carboxy terminus as a result of a 52 bp insert in exon 5, and is smaller because it is not glycosylated. Chew et al (1995) and Yang et al (1996) did not use the term MGF, but rather IGF-I Ec, to define the 4-5-6 splice variant. The muscle isoform that has the Ec domain is now known as MGF (cf McKoy et al ( 1999)). It is now clear that the particular form of the IGF-I Ec is produced by cardiac and skeletal muscle but only when they are subjected to mechanical activity.

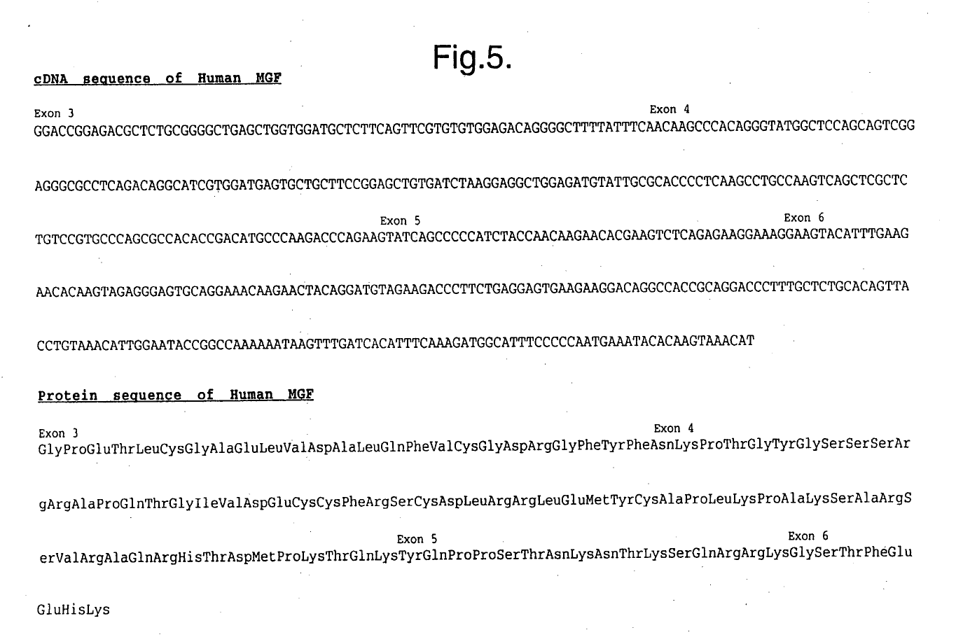

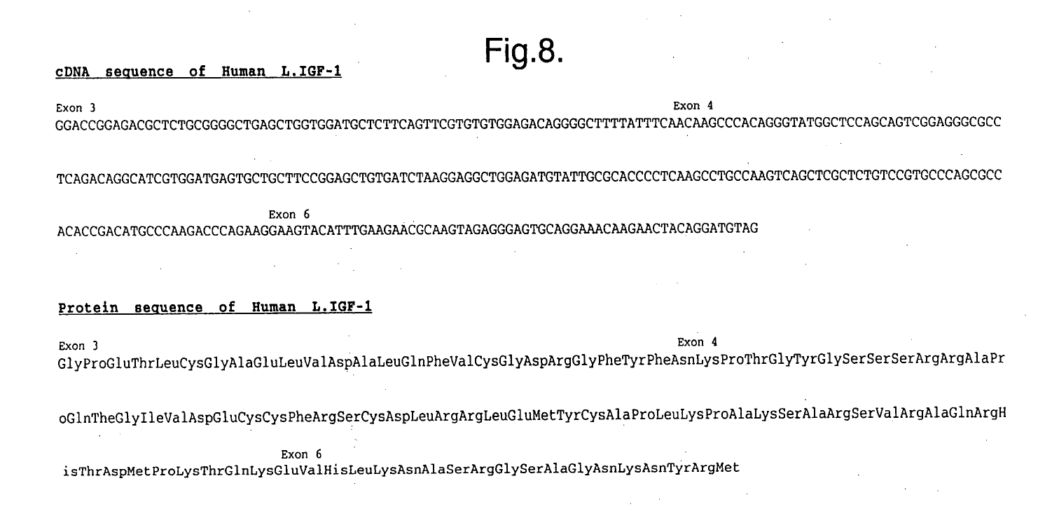

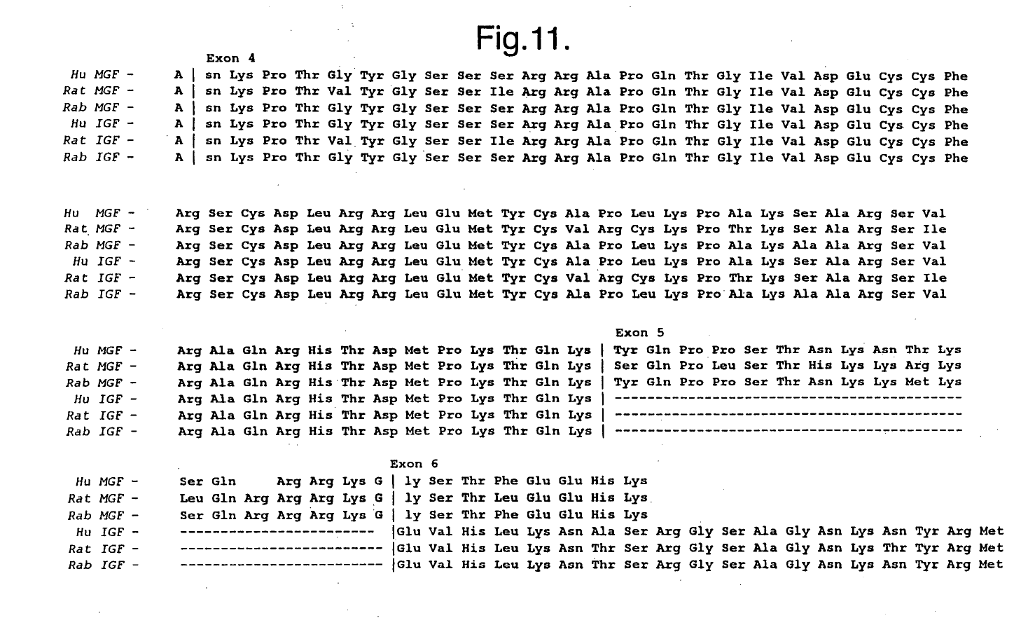

Herein, MGF is understood to mean any IGF-I polypeptide having the 4-5-6 exon structure and the neurological properties identified by the Inventors, as discussed further below. The exon structure of MGF in human, rat and rabbit is illustrated in FIGS. 5, 6 and 7 (SEQ ID NOs. 1/2, 3/4 and 5/6). For comparison, the exon structure of human, rat and rabbit L.IGF-I is given in FIGS. 8, 9 and 10 (SEQ ID NOs. 9/10, 11/12 and 13/14), and a comparison between MGF and L-IGF-I is made in FIG. 11.

Preferably, MGF of the invention will have the reading frame which, in native MGF, is generated by the 52 bp insert mentioned above. Preferably, MGF of the invention will not be glycosylated. However, it may be glycosylated or partially glycosylated in some embodiments. By partially glycosylated is meant up to 10, 20, 30, 50, 70, 80, 90, 95 or 99% as much glycosylation as L.IGF-I, e.g. containing some, but not all, of IGF-I's glycosylation sites. The pattern of glycosylation may be the same as that of L.IGF-I in terms of the type and placement of sugars or it may be different.

Preferably, MGFs of the invention comprise exons 3, 4, 5 and 6 on equivalent sequences. Optionally, they may include exons 1 and/or 2, or equivalent sequences as well.

MGF of the invention may find its origins in any species that has 4-5-6 spliced IGF-I. Thus, MGF of the invention may have the sequence of human MGF, which is generally preferred. MGF having the sequence of an animal MGF may also be used, e.g. rat, rabbit, mouse, cow, sheep, goat, chicken, dog, cat MGF. Preferably, the species origin of the MGF used will be matched to the species of the subject to be treated. In particular, it is preferred to use human MGF to treat human patients.

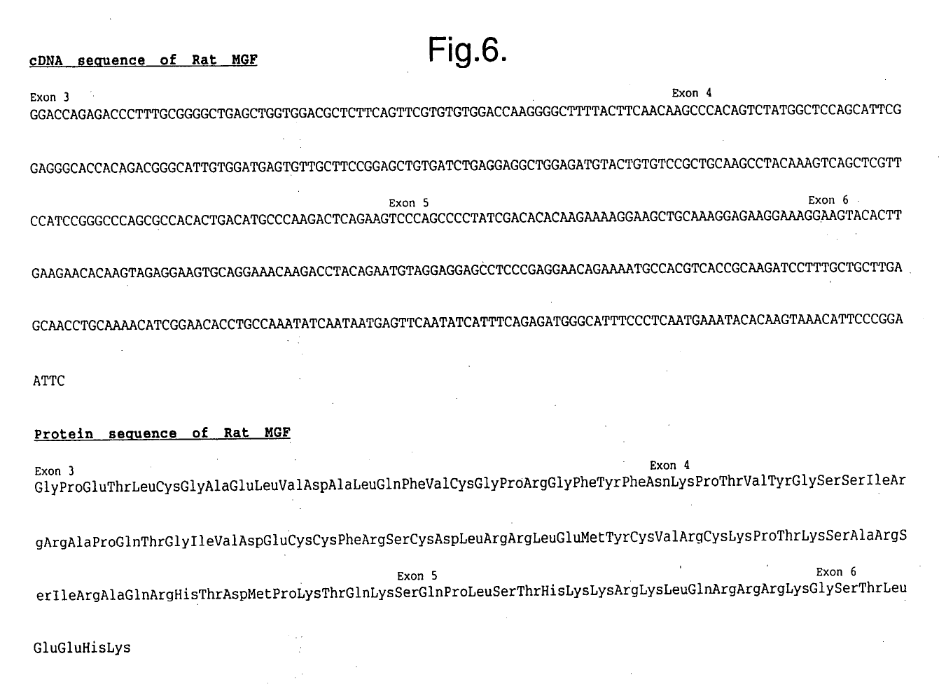

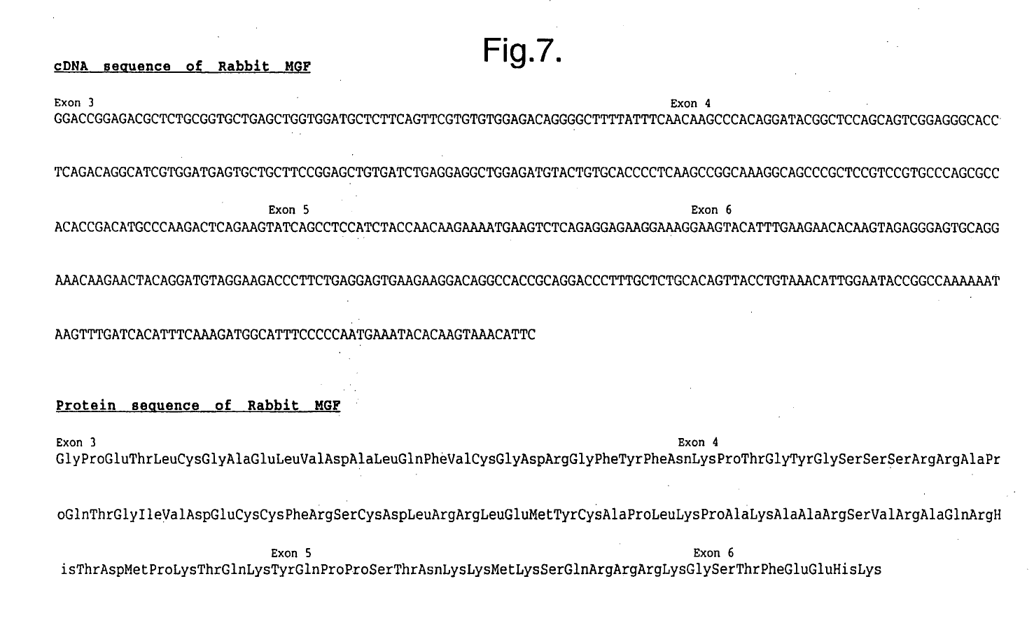

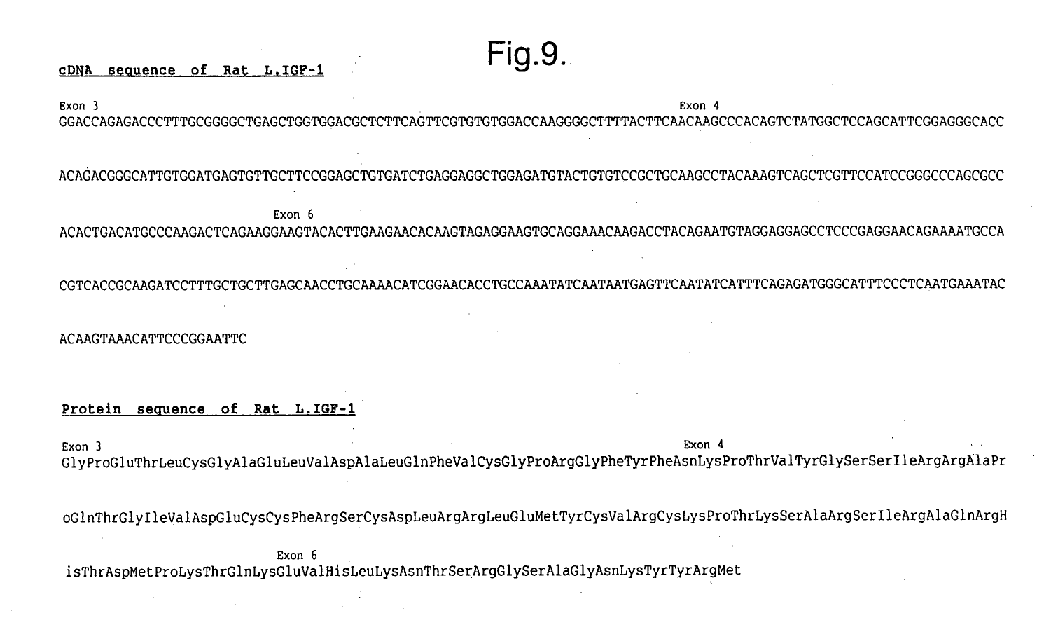

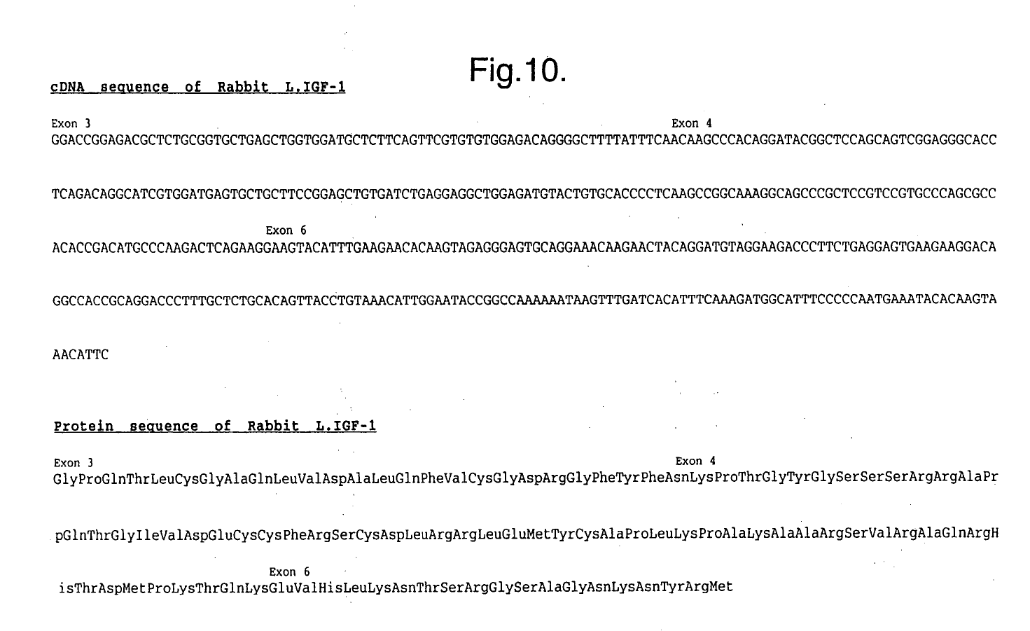

The sequences of exons 3, 4, 5 and 6 human MGF (IGF-I-Ec) (SEQ D NO. 1/2, FIG. 5), rat MGF (SEQ D NO. 3/4, FIG. 6) and rabbit MGF (IGF-I Eb) (SEQ ID NO. 5/6, FIG. 7) are given below, together with their corresponding cDNA sequences. SEQ ID NOs. 1, 3 and 5 are the cDNAs; SEQ ID NOs. 2, 4 and 6 are the polypeptides. For comparison, the sequences of exons 3, 4 and 6 human (SEQ ID NO. 9/10, FIG. 8), rat (SEQ ID NO. 11/12, FIG. 9) and rabbit (SEQ ID NO. 13/14, FIG. 10) liver-type IGF-I (L.IGF-I) are also given (see FIG. 11 in particular for comparison). Polypeptides having the sequences of SEQ ID NOs. 2, 4 and 6 may be used in preferred embodiments of the invention.

Herein, MGF and functional equivalents thereof have the neurological properties identified by the Inventors. Thus, they have the capacity to effect motoneurone rescue. The exact degree of motoneurone rescue will vary from case to case, depending on which MGF is used and under what circumstances. However, with reference to the Examples, MGFs of the invention may be able to reduce motoneurone loss by up to 20, 30, 40, 50, 60, 70, 80, 90, 95, 99 or 100% in a treated subject compared to an equivalent situation in a non-treated subject. Reduction of motoneurone loss by 70% or more, or 80% more (i.e. to 30% or less or 20% or less) is preferred. The degree of rescue may be calculated using any suitable technique, e.g. a known technique such as Stereology (see the Examples). As a specific test, the techniques used in the Exanples, which rely on measuring motoneurone rescue in response to facial nerve avulsion in rats, may be used. However it will be appreciated that this technique may not be ideal for assessing the properties non-rat MGFs. Similar tests may thus be devised using other animal models. For example, tests relating to avulsion of other nerves may be devised. So far as human treatments are concerned, it will generally be necessary to rely on animal models so human MGF may have lower activity in these models than it has in vivo in humans.

MGFs having the sequence of naturally occurring MGFs are preferred. However, variant MGFs having the same basic 4-5-6 exon structure and neurological properties discussed herein may also be used.

Polypeptides of the invention may be encoded by polynucleotides as described below.

An MGF polypeptide of the invention may consist essentially of the amino acid sequence set out in SEQ ID NO. 2, 4 or 6 or a substantially homologous sequence, or of a fragment of either of these sequences, as long as the neurological properties of the invention are maintained. In general, the naturally occurring amino acid sequences shown in SEQ ID NOs. 2, 4 and 6 are preferred. However, the polypeptides of the invention include homologues of the natural sequences, and fragments of the natural sequences and of their homologues, which have the neurological properties of the invention.

In particular, a polypeptide of the invention may comprise:
(a) the polypeptide sequence of SEQ ID NO. 2 (human MGF), 4 (rat MGF), or 6 (rabbit MGF);
(b) a polypeptide sequence at least 70, 80, 90, 95, 98 or 99% homologous to, a polypeptide of (a);
(c) a sequence comprising the amino acids encoded wholly or partly by exons 4, 5 and 6 of human, rat or rabbit MGF DNA of SEQ ID NO. 1, 3, or 5, or a sequence having 70% or greater homology thereto;
(d) a sequence encoded by a nucleic acid sequence capable of selectively hybridising to a sequence of (a), (b) or (c); or
(e) an allelic variant or species homologue of a sequence of (a).

Allelic Variants

An allelic variant will be a variant which occurs naturally and which will function in a substantially similar manner to the protein of SEQ ID NO. 2, 4 or 6 as defined above. Similarly, a species homologue of the protein will be the equivalent protein which occurs naturally in another species. Such a homologue may occur in any species, preferably a mammalian species, for example a bovine, equine, ovine, feline or canine species; such as cow, horse, sheep or goat, cat, or dog; or in a rodent species other than rat (SEQ ID NO. 4) or rabbit (SEQ ID NO. 6), or in a primate species other than human (SEQ D NO. 2). Non-mammalian MGFs, for example piscine or avian MGFs, e.g. chicken MGF, are also MGFs of the invention. Within any one species, a homologue may exist as several allelic variants, and these will all be considered homologues of the protein of SEQ ID NO. 2, 4 or 6.

Allelic variants and species homologues can be obtained by methods known in the art, e.g. by probing suitable cell source with a probe derived from SEQ ID NO. 1, 3 or 5. Clones obtained can be manipulated by conventional techniques to generate a polypeptide of the invention which can be produced by recombinant or synthetic techniques known per se.

Homologues

A polypeptide of the invention is preferably at least 70% homologous to the protein of SEQ ID NO. 2, 4 or 6 more preferably at least 80 or 90% and more preferably still at least 95, 97 or 99% homologous thereto over a region of at least 20, preferably at least 30, for instance at least 40, 60 or 100 or more contiguous amino acids. Methods of measuring protein homology are well known in the art and it will be understood by those of skill in the art that in the present context, homology is calculated on the basis of amino acid identity (sometimes referred to as “hard homology”).

Degrees of homology can be measured by well-known methods, as discussed herein for polynucleotide sequences.

The sequence of the polypeptides of SEQ ID NOs. 2, 4 and 6 and of the allelic variants and species homologues can be modified to provide further polypeptides of the invention.

Substitutions

Amino acid substitutions may be made, for example from 1, 2 or 3 to 10, 20 or 30 substitutions. For example, a total of up to 1, 2, 5, 10 or 20 amino acids may be substituted over a length of 50, 100 or 200 amino acids in the polypeptides. For example, up to 20 amino acids substituted over any length of 50 amino acids. The modified polypeptide generally retains the neurological properties of the invention, as defined herein. Conservative substitutions may be made, for example according to the following table. Amino acids in the same block in the second column and preferably in the same line in the third column may be substituted for each other.

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Fragments

Polypeptides of the invention also include fragments of the above-mentioned full length polypeptides and variants thereof, including fragments of the sequence set out in SEQ ID NOs. 2, 4 and 6. Such fragments typically retain the neurological properties of the invention.

Suitable fragments will generally be at least about 20, e.g. at least 20, 50 or 100 amino acids in size. Polypeptide fragments of the polypeptides of SEQ ID NOs. 2, 4 and 6 and allelic and species variants thereof may contain one or more (e.g. 2, 3, 5, 5 to 10 or more) substitutions, deletions or insertions, including conservative substitutions. Each substitution, insertion or deletion may be of any length, e.g. 1, 2, 3, 4, 5, 5 to 10 or 10 to 20 amino acids in length.

In particular, fragments of the invention may comprise the amino acids encoded by exons 4, 5 and 6 of human, rat or rabbit DNA of SEQ ID NO. 1, 3 or 5. The first amino aid of exon 4, Asn, is partly encoded by exon 3 (1 nucleotide) and partly by exon 4 (2 nucleotides). It is preferred that said first amino acid be present, in a fragment of the invention.

Chimeric Sequences

MGF polypeptides encoded by chimeric polypeptide sequences of the invention (see below) may be used.

Isolation, Purification and Modification

Polypeptides of the invention may be in a substantially isolated form. It will be understood that the polypeptide may be mixed with carriers or diluents which will not interfere with the intended purpose of the polypeptide and still be regarded as substantially isolated. A polypeptide of the invention may also be in a substantially purified form, in which case it will generally comprise the polypeptide in a preparation in which more than 70%, e.g. more than 80, 90, 95, 98 or 99% of the polypeptide in the preparation is a polypeptide of the invention.

Polypeptides of the invention may be provided in a form such that they are outside their natural cellular environment. Thus, they may be substantially isolated or purified, as discussed above, or in a cell which they do not occur in nature, e.g. a cell or other plant species, animals, yeast or bacteria.

Polypeptides of the invention may be modified for example by the addition of Histidine residues or a T7 tag to assist their identification or purification or by the addition of a signal sequence to promote their secretion from a cell.

A polypeptide of the invention may be labelled with a revealing label. The revealing label may be any suitable label which allows the polypeptide to be detected. Suitable labels include radioisotopes, e.g. ¹²⁵I, ³⁵S, enzymes, antibodies, polynucleotides and linkers such as biotin.

Polypeptides of the invention may be chemically modified, e.g. post-translationally modified. For example, they may comprise modified amino acid residues. They may also be glycosylated (see above), though MGF is not naturally glycosylated. Such modified polypeptides will be understood to be polypeptides of the invention.

Another possibility is to increase the stability, and hence half life of MGF in vivo by altering its sequence, e.g. to make it more amenable to glycosylation by introducing one or more glycosylation sites. Alternatively, modifications can be made that make MGF's primary amino acid structure more resistant to degradation.

The effects of modifications to MGF's sequence can be tested by anv suitable method. For example, the binding properties and/or stability of variant MGFs can be tested by comparing them in vitro or in vivo to those of unmodified MGF.

Polynucleotides

Polynucleotides of the invention encode polypeptides of the invention.

Preferred polynucleotides of the invention comprise a coding sequence encoding a polypeptide having the neurological properties of the invention, which coding sequence is selected from:
(a) the coding sequence of any one of SEQ ID NO. 1, 3 or 5,
(b) a sequence capable of selectively hybridising to a sequence of (a), or to a sequence complementary to a sequence of (a);
(c) a sequence having 70% or more homology to a sequence of (a);
(d) a sequence which is a fragment of the sequence of any one of (a) to (c); and
(e) a sequence which differs from that of any one of (a) to (d) but which, owing to the degeneracy of the genetic code, encodes the same polypeptide.

Thus, the invention provides polynucleotides comprising the coding sequence as shown in any one of SEQ ID NO. 1, 3 or 5 and variants thereof with related sequences. Polynucleotides of the invention can be used to prepare vectors of the invention.

SEQ ID NOs. 1, 3 and 5

Preferred polynucleotides of the invention comprise coding sequences as shown in SEQ ID NOs. 1, 3 and 5.

Hybridisable Sequences

A polynucleotide of the invention may hybridise selectively to coding sequence of SEQ ID NO. 1, 3 or 5 at a level significantly above background. Background hybridisation may occur, for example because of other cDNAs present in a cDNA library. The signal level generated by the interaction between a polynucleotide of the invention and the coding sequence of SEQ ID NO. 1, 3, 5, 7, 9 or 11 is typically at least 10 fold, preferably at least 100 fold, as intense as interactions between other polynucleotides and the coding sequence of SEQ ID NO. 1, 3 or 5. The intensity of interaction may be measured, for example by radiolabelling the probe, e.g. with ³²P. Selective hybridisation is typically achieved using conditions of medium to high stringency (for example 0.03M sodium chloride and 0.03M sodium citrate at from about 50° C. to about 60° C., for example 45 to 50, 50 to 55 or 55 to 60° C., e.g. at 50 or 60° C.

However, such hybridisation may be carried out under any suitable conditions known in the art (see Sambrook et al (1989), Molecular Cloning: A Laboratory Manual).

For example, if high stringency is required, suitable conditions include 0.2×SSX at around 60° C., for example 40 to 50° C., 50 to 60° C. or 60 to 70° C., e.g. at 50 or 60° C. If lower stringency is required, suitable conditions include 2×SSC at around 60° C., for example 40 to 50° C., 50 to 60° C. or 60 to 70° C., e.g. at 50 or 60° C.

Stringency typically occurs in a range from about Tm-5° C. (5° C. below the melting temperature (Tm) of the two sequences hybridising to each other in a duplex) to about 20° C. to 25° C. below Tm. Thus, according to the invention, a hybridisable sequence may be one which hybridises to SEQ ID NO. 1, 3 or 5 at a temperature of from Tm to Tm-25° C., e.g. Tm to Tm-5° C., Tm-5 to Tm-10° C., Tm-10 to Tm-20° C. or Tm-20 to Tm-25° C.

Homologous Sequences

A polynucleotide sequence of the invention, will comprise a coding sequence at least 70% preferably at least 80 or 90% and more preferably at least 95, 98 or 99%, homologous to the coding sequence of SEQ ID NO. 1, 3 or 5.

Such homology will preferably apply over a region of at least 20, preferably at least 50, for instance 100 to 500 or more, contiguous nucleotides.

Methods of measuring nucleic acid and polypeptides homology are well known in the art. These methods can be applied to measurement of homology for both polypeptides and nucleic acids of the invention. For example, the UWGCG Package provides the BESTFIT program which can be used to calculate homology (Devereux et al (1984), Nucleic Acids Research 12, p. 387-395).

Similarly, the PILEUP and BLAST algorithms can be used to line up sequences (for example as described in Altschul, S. F. (1993) J. Mol. Evol. 30:290-300; Altschul, S. F. et al (1990) J. Mol. Biol. 215:403-410).

Many different settings are possible for such programs. According to the invention, the default settings may be used.

In more detail, the BLAST algorithm is suitable for determining sequence similarity and it is described in Altschul et al (1990) J. Mol. Biol. 215:403-410). Software for performing BLAST analyses is publicly available through the National Center for Biotechnology Information (http://www.ncbi/nlm.bih.gov/). This algorithm involves first identifying high scoring sequence pair (HSPs) by identifying short words of length W in the query sequence that either match or satisfy some positive-valued threshold score T when aligned with a word of the same length in a database sequence. T is referred to as the neighbourhood word score threshold (Altschul et al, supra). These initial neighbourhood word hits act as seeds for initiating searches to find HSPs containing them. The word hits are extended in both directions along each sequence for as far as the cumulative alignment score can be increased. Extensions for the word hits in each direction are halted when: the cumulative alignment score falls off by the quantity X from its maximum achieved value, the cumulative score goes to zero or below, due to the accumulation of one or more negative-scoring residue alignments; or the end of either sequence is reached. The BLAST algorithm parameters W, T and X determine the sensitivity and speed of the alignment. The BLAST program uses as defaults a word length (W) of 11, the BLOSUM62 scoring matrix (see Henikoff and Henikoff (1992) Proc. Natl. Acad. Sci. USA 89:10915-10919) alignments (B) of 50, expectation (E) of 10, M=5, N=4, and a comparison of both strands.

The BLAST algorithm performs a statistical analysis of the similarity between two sequences; see e.g. Karlin and Altschul (1993) Proc. Natl. Sci. USA 90:5873-5787. One measure of similarity provided by the BLAST algorithm is the smallest sum probability (P(N)), which provides an indication of the probability by which a match between two nucleotide or amino acid sequences would occur by chance. For example, a nucleic acid is considered similar to a fused gene or cDNA if the smallest sum probability in comparison of the test nucleic acid to a fused nucleic acid is less than about 1, preferably less than about 0.1, more preferably less than about 0.01, and most preferably less than about 0.001.

Fragments

Also included within the scope of the invention are sequences which are fragments of the sequences of (a) to (c) above but have the neurological properties of the invention.

In particular, fragments may comprise exons 4, 5 and 6 of human, rat or rabbit MGF DNA of SEQ ID NO. 1, 3 or 5.

The first amino acid of exon 4, Asn, is partly encoded by exon 3 and partly by exon 4. It is preferred that the necessary coding bases from exon 3 are present to encode said first amino acid, Asn.

Degenerate Sequences

Also included within the scope of the invention are sequences that differ from those of (a) to (d) but which, because of the degeneracy of the genetic code, encode the same protective polypeptides. For example, the invention provides degenerate variants of the sequence of SEQ ID NOs. 1, 3 and 5 that also encode the polypeptide of SEQ ID NOs. 2, 4 and 6.

Complementary Sequences

In addition, the invention provides polynucleotides having sequences complementary to any of the above-mentioned sequences.

Chimeric Sequences

Chimeric sequences comprising exons from more than one species may also be used. For example, one or more of exons 3 to 6 may be derived from human and one or more from rat and/or rabbit.

Further Properties

The nucleic sequences of the invention may be of any length as long as they encode a polypeptide of the invention. A nucleic acid sequence according to the invention may be a contiguous fragment of the sequence of SEQ ID NO. 1, 3 or 5 or a sequence that is related to it in any of the ways described above. Alternatively, nucleic acids of the invention may comprise DNA sequences that are not contiguous in the sequence of SEQ ID NO. 1, 3 or 5. These sequences may be fragments of the sequence of SEQ ID NO. 1, 3 or 5 or nucleic acid sequences that are related to such fragments in any of the ways described above. Nucleic acid sequences of the invention will preferably comprise at least 50 bases or base pairs, for example 50 to 100, 100 to 500, 500 to 1000 or 1000 to 2000 bases or base pairs.

Any combination of the above-mentioned degrees of homology and minimum sizes may be used to defined polynucleotides of the invention, with the more stringent combinations (e.g. higher homology over longer lengths and/or hybridisation under more stringent conditions)being preferred. Thus, for example a polynucleotide which is at least 90% homologous over 100, preferably over 200 nucleotides forms one aspect of the invention, as does a polynucleotide which is at least 95% homologous over 100 or 200 nucleotides.

Polynucleotides of the invention may comprise DNA or RNA. They may also be polynucleotides which include within them synthetic or modified nucleotides. A number of different types of modification to polynucleotides are known in the art. Modifications may, for example enhance resistance to nucleases and/or enhance ability to enter cells. For example, phosphorothioate oligonucleotides may be used. Other deoxynucleotide analogs include methylphosphonates, phosphoramidates. phosphorothioates, N3′P5′-phosphoramidates and oligoribonucleotide phosphorothioates and their 2′-O-alkyl analogs and 2′-O-methyliribonucleotide methylphosphonates. A further possible modification is the addition of acridine or polylysine chains at the 3′ and/or 5′ ends of the molecule.

Alternatively mixed backbone oligonucleotides (MBOs) may be used. MBOs contain segments of phosphothioate oligodeoxynucleotides and appropriately placed segments of modified oligodeoxy- or oligoribonucleotides. MBOs have segments of phosphorothioate linkages and other segments of other modified oligonucleotides, such as methylphosphonate, which is non-ionic, and very resistant to nucleases or 2′-O-alkyloliogoribonucleotides. For the purposes of the present invention, it is to be understood that the polynucleotides described herein may be modified by any method available in the art. Such modifications may be carried out in order to enhance the in vivo activity or lifespan of polynucleotides of the invention.

Polynucleotides of the invention may be used to produce a primer, e.g. a PCR primer, a primer for an alternative amplification reaction, a probe, e.g. labelled with a revealing label by conventional means using radioactive or non-radioactive labels or the polynucleotides may be cloned into vectors. Such primers, probes and other fragments will preferably be at least 10, preferably at least 15 or 20, for example at least 25, 30 or 40 nucleotides in length. These will be useful in identifying species homologues and allelic variants as discussed above.

Polynucleotides such as a DNA polynucleotides and primers according to the invention may be produced recombinantly, synthetically, or by any means available to those of skill in the art. They may also be cloned by standard techniques. The polynucleotides are typically provided in isolated and/or purified form.

In general, primers will be produced by synthetic means, involving a stepwise manufacture of the desired nucleic acid sequence one nucleotide at a time. Techniques for accomplishing this using automated techniques are readily available in the art.

Genomic clones corresponding to the cDNAs of SEQ ID NOs. 1, 3 and 5 containing, for example introns and promoter regions are also aspects of the invention and may also be produced using recombinant means, for example using PCR (polymerase chain reaction) cloning techniques.

The 4-5-6 exon pattern of MGF is characteristic of polynucleotides of the invention. Any suitable method may be used to ensure that this pattern is reflected in the coding sequence, and thus in the encoded polypeptide. For example, cDNA sequences lacking introns and splice signals and including the coding sequences of exons 4, 5 and 6 may be used. Alternatively, genomic DNA may be used if it will be correctly spliced in the situation at hand.

Although in general the techniques mentioned herein are well known in the art, reference may be made in particular to Sambrook et al (1989), Molecular Cloning: A Laboratory Manual.

Polynucleotides which are not 100% homologous to the sequences of the present invention but fall within the scope of the invention, as described above, can be obtained in a number of ways, for example by probing cDNA or genomic libraries from other plant species with probes derived from SEQ ID NO. 1, 3 or 5. Degenerate probes can be prepared by means known in the art to take into account the possibility of degenerate variation between the DNA sequences of SEQ ID NO. 1, 3 or 5 and the sequences being probed for under conditions of medium to high stringency (for example 0.03M sodium chloride and 0.03M sodium citrate at from about 50° C. to about 60° C.), or other suitable conditions (e.g. as described above).

Allelic variants and species homologues may also be obtained using degenerate PCR which will use primers designed to target sequences within the variants and homologues encoding likely conserved amino acid sequences. Likely conserved sequences can be predicted from aligning the amino acid sequences of the invention (SEQ ID NO. 2, 4 or 6) with each other and/or with those of any homologous sequences known in the art. The primers will contain one or more degenerate positions and will be used at stringency conditions lower than those used for cloning sequences with single sequence primers against known sequences.

Alternatively, such polynucleotides may be obtained by site-directed mutagenesis of sequences of SEQ ID NO. 1, 3 or 5 or allelic variants thereof This may be useful where, for example silent codon changes are required to sequences to optimise codon preferences for a particular host cell in which the polynucleotide sequences are being expressed. Other sequences may be desired in order to introduce restriction enzyme recognition sites, or to alter the properties or function of the polypeptides encoded by the polynucleotides.

The invention further provides double stranded polynucleotides comprising a polynucleotide of the invention and its complement.

Polynucleotides, probes or primers of the invention may carry a revealing label. Suitable labels include radiosotopes such as ³²P ro ³⁵S, enzyme labels, or other protein labels such as biotin. Such labels may be added to polynucleotides, probes or primers of the invention and may be detected using techniques known per se.

Delivery of MGF to Subjects

MGF and functionally equivalent polypeptides of the invention can be delivered to subjects in need of treatment for neurological disorders by any suitable method. They can be delivered directly, as polypeptides. However, delivery by means of a vector containing a nucleic acid encoding the polypeptide, which is then expressed in vivo, is preferred in some situations. MGF has a short half-life and may be most effective when provided locally at the neuromuscular junction. In vivo expression of MGF facilitates localisation and avoids the need for repeated injection.

Nevertheless, there are clinical situations in which peptide delivery will be preferred. For example, in the case of a stroke or alcohol-related brain damage when neuronal cell death must be prevented as soon as possible, peptide delivery would be preferred. One possibility is to deliver the polypeptide directly in the first instance, e.g. immediately after an injury, then rely on expression of the polypeptide in vivo for long, term therapy.

Delivery of Polypeptides

Polypeptides of the invention may be delivered by any suitable means. Intravenous delivery is a preferred option. Owing to MGF's short half-life, slow-release on delivery agents may be used. Any suitable pharmaceutical formulation may be used to effect slow-release of MGF of the invention. Liposome formulations are one possibility.

Production of Polypeptides

Polypeptides of the invention may be produced in any suitable manner. In some embodiments they may be extracted from animal tissues. However, it is preferred that they be produced recombinantly. This can be done using known techniques.

Delivery of Nucleic Acids

Delivery by means of a vector containing a nucleic acid encoding the polypeptide, which is then expressed in vivo, is preferred in some situations. MGF has a short half-life and may be most effective when provided locally at the neuromuscular junction. In vivo expression of MGF facilitates localisation and avoids the need for repeated injection.

Vectors for Delivery of Nucleic Acids

The nucleic acids of the invention may be delivered in any suitable manner. In particular, they will generally be delivered via a vector. Any suitable type of vector may be used.

The nucleic acid may be delivered in a “naked” form (e.g. in a plasmid vector), optionally associated with an agent to assist in its penetration, as discussed below. Alternatively, the vector may be one that encapsulates the nucleic acid, e.g. a virus.

The vector may, for example be a plasmid or cosmid vector.

The vector may be a viral vector, such as a vector comprising a virus able to infect the cells of the recipient subject. Thus, the vector may be, or may be derived from any suitable virus, for example an alphavirus, adenovirus, adeno-associated virus, baculovirus, vaccinia virus, herpes virus, herpes simplex virus, retrovirus (e.g. lentivirus) vector, or baculovirus. A virus vector will be disabled, in the sense that it will not typically be able to replicate or cause pathological effects in the same way as on intact virus. It will typically be attenuated, for example replication defective.

Especially when it is delivered in a “naked” form, e.g. as a plasmid, the polynucleotide may be associated with an agent to assist in penetration of cells. Examples include cationic agents (e.g. catioric lipids), polylysine, lipids, and precipitating agents (e.g. a calcium salt). Such agents generally aid the passage of the polynucleotide across the cell membrane. The polynucleotide may be in the form of liposomes or particles, for example in association with any of the penetrating agents mentioned above. The polynucleotide may be in association with an agent that causes the polynucleotide to adopt a more compact form, such as a histone. The polynucleotide may be in association with spermidine.

Similarly, liposomes may be used to help transport polynucleotides of the invention into cells.

The polynucleotide may be associated with a carrier which can be used to deliver the polynucleotide into the cell, or even into the nucleus, using biolistic techniques. Such a carrier may be a metal particle, such as a cold or tungsten particle.

The polynucleotide is typically capable of being expressed in a cell of the recipient. Thus, the polynucleotide typically also comprises control sequences which are operably linked to the MGF coding sequence of the invention, said control sequences being capable of expressing the coding sequence in the cells of the recipient, for example after integration of the polynucleotide into the genome of the cell.

The control sequences typically comprise a promoter (generally 5′ to the coding sequence) and/or a terminator and/or translation initiation sequence (e.g. GCCACCATGG (SEQ ID NO. 7) or GCCCCCATGG (SEQ ID NO. 8)) and/or a translational stop codon (e.g. TAA, TAG or TGA) and/or a polyadenylation signal and/or one or more enhancer sequences and/or a RNA pause site. The control sequences may enhance the transcription or translation of the polynucleotide. The control sequences may be tissue-specific so that the polynucleotide is only expressed in certain tissues, or may be the control sequences of a constitutively expressed gene. Muscle-specific promoters and enhancers are particularly preferred. The control sequences are typically those of any of the eukaryotes mentioned herein or of a virus which infects a eukaryote, e.g. of the species of the recipient, such as a human virus for a human recipient. The polynucleotide may comprise an origin of replication.

The promoter may, for example be (in particular for expression in mammalian cells) a metallothionein gene promoter, SV40 large T antigen promoter, CMV or adenoviral promoter.

So far as tissue-specific expression is concerned, muscle-specific control elements, such as muscle-specific promoters and enhancers, are particularly preferred, especially where the nucleic acid is to be delivered intramuscularly, e.g. in plasmid form. Such elements can be derived from, for example myosin genes. For example, myosin light chain or heavy chain promoters may be used, as may myosin light chain or heavy chain enhancers.

Several myosin enhancers and promoters have been identified to date from both myosin light chain and myosin heavy chain genes. Preferably, the myosin enhancer and/or promoter used is of vertebrate origin, more preferably avian, piscine or mammalian origin.

A myosin light chain enhancer is preferred. A rat myosin light chain 1/3 enhancer (Donoghue et al (1988) Genes Dev. 2:1779-1790; Neville et al (1996) Dev. Genetics 19:157-162) is especially preferred. The enhancer is operably linked to the promoter. The enhancer may be either upstream or downstream of the promoter. The enhancer may be used in either orientation.

A myosin heavy chain promoter is preferred. A particularly preferred myosin heavy chain promoter is a truncated rabbit β-cardiac myosin heavy chain promoter, in particular up to and including 789 base pairs upstream of the transcriptional start site. Another myosin heavy chain promoter which is preferred is the carp FG2 promoter, in particular up to and including 901 base pairs upstream of the transcription start site (Gauvry etal(1996) Eur. J. Biochem. 236:887-894). Further details of myosin heavy chain promoters derived from rat, rabbit, human, porcine and chick myosin heavy chain genes are given in Gauvry et al (1996) and references therein. All of these promoters may be used in the present invention.

In this context, introduction of MGF of the invention may be linked with physical activity. As muscles respond to exercise and myosin is the most abundant protein in muscle the myosin promoter/enhancer regulatory elements means that the expression of the cDNA will be upregulated by increased muscular activity.

Plasmid vectors and disabled viral vectors are preferred embodiments. Plasmid vectors are particularly preferred, especially for intramuscular administration aimed at securing local expression in the muscle.

The vector may be designed for stable integration into the genome of the recipient's cells. Alternatively, it may be designed to be non-integrative. In stable introduction the polynucleotide becomes integrated into the genome of the cell (i.e. becomes contiguous with genome). Thus, the polynucleotide may also comprise a sequence which enhances integration of the polynucleotide such as the loxP sites of the bacteriophage P1 Cre recombination system, FRT sites of the yeast FLP recombination system or Adeno-associated virus (AAV) terminal repeat sequences. Integration may be enhanced by other factors which are present, such as bacteriophage P1 derived Cre, yeast derived FLP recombinase, AAV Rep proteins, Cre or FLP recombinases or bacterial Rec proteins. In one embodiment, the polynucleotide of the invention is capable of expressing such a factor.

The polynucleotide may be one which integrates randomly (such as in a non-sequence specific manner) into any position in the genome or one which preferentially integrates at particular sites of the genome. Generally the whole coding sequence of the polynucleotide and the control sequences will be present in the genome after integration.

Pharmaceutical Compositions and Formulations

The polypeptides and nucleic acids of the invention are preferably delivered in the form of a pharmaceutical formulation comprising a pharmaceutically acceptable carrier or diluent. Any suitable pharmaceutical formulation may be used.

For example, suitable formulations may include aqueous and non-aqueous sterile injection solutions which may contain anti-oxidants, buffers, bacteriostats, bactericidal antibiotics and solutes which render the formulation isotonic with the bodily fluids of the intended recipient; and aqueous and non-aqueous sterile suspensions which may include suspending agents and thickening agents. The formulations may be presented in unit-dose or multi-dose containers. For example, sealed ampoules and vials, and may be stored in a frozen or freeze-dried (lyophilized) condition requiring only the addition of the sterile liquid carrier, for example water for injections, immediately prior to use.

It should be understood that in addition to the ingredients particularly mentioned above the formulations of this invention may include other agents conventional in the art having regard to the type of formulation in question. Sterile, pyrogen-free aqueous and non-aqueous solutions are preferred.

Routes of Administration

Polypeptides and nucleic acids of the invention may be administered by any suitable form of administration, for example topical, cutaneous, parenteral, intramuscular, subcutaneous or transdermal administration, or by direct injection into the bloodstream or by direct application to mucosal tissues. Intramuscular administration is preferred for plasmids and other naked nucleic acids.

Dosages

The proteins, nucleic acids and vectors of the invention may be delivered in any suitable dosage, and using any suitable dosage regime. Persons of skill in the art will appreciate that the dosage amount and regime may be adapted to ensure optimal treatment of the particular condition to be treated, depending on numerous factors. Some such factors may be in the age, sex and clinical condition of the subject to be treated.

The dosage used for the delivery of nucleic acids by vectors will depend on many factors, including the efficiency with which the vectors deliver the nucleic acids to cells, and the efficiency with which the nucleic acids are expressed in the cells.

For the delivery of naked nucleic acids (e.g. plasmids or other naked non-viral vectors), typical doses are from 0.1 to 5000 μg, for example 10 to 1000 μg, such as 10 to 100 μg, 100 to 500 μg and 500 to 2000 μg per dose.

As a guide, viral vectors may be delivered in doses of from 10⁴ to 10¹⁴ cfu or pfu/ml, for example 10⁴ to 10⁶, 10⁶ to 10⁸, 10⁸ to 10¹⁰, 10¹⁰ to 10¹² or 10¹² to 10¹⁴ cfu or pfu/ml. Doses in the region of 10⁵ to 10⁹ cfu or pfu/ml are preferred. The term pfu (plaque forming unit) applies to certain viruses, including adenoviruses, and corresponds to the infectivity of a virus solution, and is determined by infection of an appropriate cell culture, and measurement, generally after 48 hours, of the number of plaques of infected cells. The term cfu (colony forming unit) applies to other viruses, including retroviruses, and is determined by means known in the art generally following 14 days incubation with a selectable marker. The techniques for determining the cfu or pfu titre of a viral solution are well known in the art.

For retroviruses, dosages in the region of 10⁵ to 10⁶ cfu/ml are particularly preferred. For pseudotyped retroviruses, dosages in the region of 10⁷ cfu/ml are particularly preferred. For adenoviruses, dosages in the region of 10⁹ pfu/ml are particularly preferred.

For the delivery of polypeptides of the invention suitable doses include doses of from 1 to 1000 μg, from 10 to 100 μg, from 100 to 500 μg and from 500 to 1000 μg.

Dosage schedules will also vary according to, for example the route of administration, the species of the recipient and the condition of the recipient. However, single doses and multiple doses spread over periods of days, weeks of months are envisaged. As discussed above, delivery by means of nucleic acids that are expressed in vivo is advantageous because it minimises the need for injections into the subject.

Neurological Disorders

As discussed above, MGF may be used to treat neurological disorders. Treatment of neurodegenerative disorders is preferred. Treatment of motoneurone disorders, especially neurodegenerative disorders of motoneurones is preferred.

Examples of disorders include amyotrophic lateral sclerosis; spinal muscular atrophy; progressive spinal muscular atrophy; infantile or juvenile muscular atrophy, poliomyelitis or post-polio syndrome; a disorder caused by exposure to a toxin, motoneurone trauma, a motoneurone lesion or nerve damage; an injury that affects motoneurones; and motoneurone loss associated with ageing; and autosomal as well as sex-linked muscular dystrophy; Alzheimer's disease; Parkinson's disease; diabetic neuropathy; and peripheral neuropathies.

Preferably, the effects of the treatment involve motoneurone rescue. Notably, the present Inventors are the first to appreciate that IGF-I is capable of effecting motoneurone rescue in intact adult motoneurones. Treatments based on adult motoneurone rescue are thus preferred.

Combinations of MGF and Other Neurotrophic Factors

MGF polypeptides and nucleic acids of the invention can be administered in combination with other neurologically active agents. Any additional neurological active agent may be used in this way. Such agents may be non-polypeptide molecules or they may be polypeptides. If they are polypeptides, they may be delivered as polypeptides or as nucleic acids encoding such polypeptides. This may be done by any suitable method, for example by a method as described herein for delivering of MGF or nucleic acids encoding MGF.

Polypeptide growth factors having neurological activity are preferred. For example, neurotophins such as Brain-Derived Neurotrophic Factor (BDNF). Neurotrophin-3 (NT-3), NT-4, NT-5 or Nerve Growth Factor (NGF) may be used. Similarly, neurologically active cytokines such as Ciliary Neurotrophic Factor (CNTF) can be used. Similarly, neurologically active transcription factors such as Brn 3a, Brn 3b and Brn 3c may be used.

When an MGF of the invention is combined with another neurotogically active agent in the treatment of a neurological disorder the two may be combined in the same pharmaceutical composition. Alternatively, they may be administered in separate compositions. They may be administered simultaneously, separately or sequentially and at the same site or a different site.

### EXAMPLES

### Introduction

In this study, we have used a model of axotomy-induced motoneuroneal degeneration in adult rats to examine the protective effects of two isoforms of insulin-like growth factor-I (IGF-I): the commonly-used liver-type isoform (L.IGF-I) and a newly-identified splice variant of IGF-I which is produced by active muscle (Yang, el al, 1996) and which we have termed “mechano growth factor” (MGF). Our analysis of the structure of MGF indicates that it probably has different tissue binding, and a shorter half-life than L.IGF-I making it particularly suited to mediating, such local interactions in a paracrine/autocrine manner. To enable the local action of L.IGF-I and MGF at the neuromuscular junction and avoid the need for repeated injections of these short half-life molecules, we used a plasmid DNA vector to deliver the genes for these growth factors to muscles.

Methods

Three 20 μl equidistant injections were made into the right whisker pad of lightly-anaesthetised (2% halothane) 6 m Sprague-Dawley rats (n=4 per group). In the first group (plasmid), 1.5 μg/μl plasmid DNA containing the rat MGF gene was injected and in the third group 0.65 μg/μl plasmid DNA containing the rat MGF gene was injected. After 7 days, the right facial nerve was avulsed as it emerged from the stylomastoid foramen using gentle traction. In other groups, the right facial nerve was crushed (n=4) or avulsed (n=4) without prior intramuscular injection of plasmid. After 1 month, all rats, including 4 non-operated rats, were anaesthetised then perfused with 4% paraformaldehyde and the region of the brainstem containing the facial nucleus sectioned serially at 70 μm using a vibratome. Every 5^{th} section was taken in a systematic random manner and stained with the fluorescent dye YOYO (1:1000, molecular probes) for estimation of total facial motoneurone number using a modification of the discetor method for use in the confocal microscope (Johnson et al, 1998). Briefly, 2 optical sections separated by 10 μm were taken through the 70 μm vibratome slice, one image was stored as shades of green and the other as shades of red. The two optical sections were then merged on screen and only those neurones which were present in one optical section but not the other (which in this case were green, but not red or shades of yellow) were counted. After determining the volume of the facial nucleus using stereology (West M. J. Trends in Neuroscience 1999. 22: 51-61) the total number of facial motoneurones was then calculated.

Results

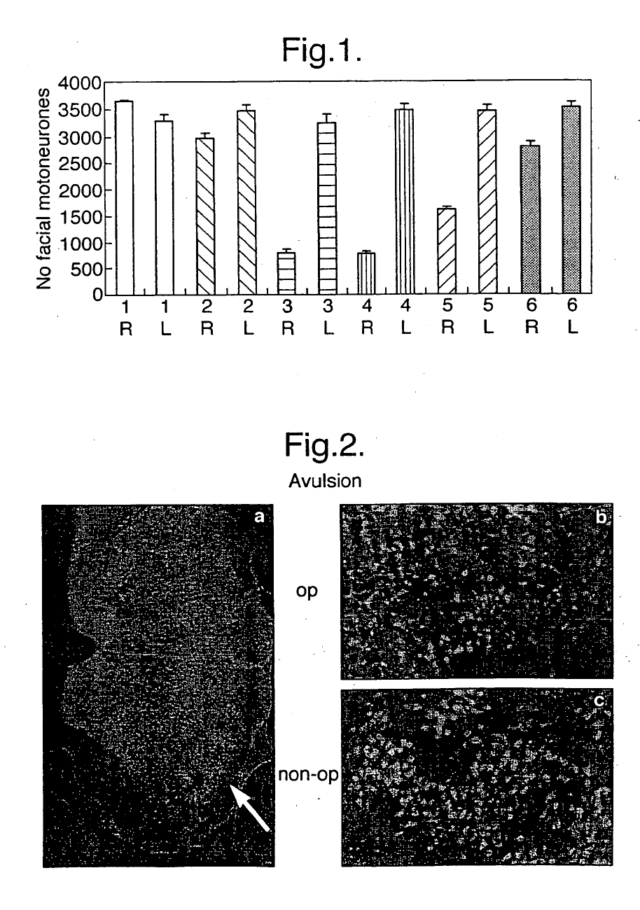

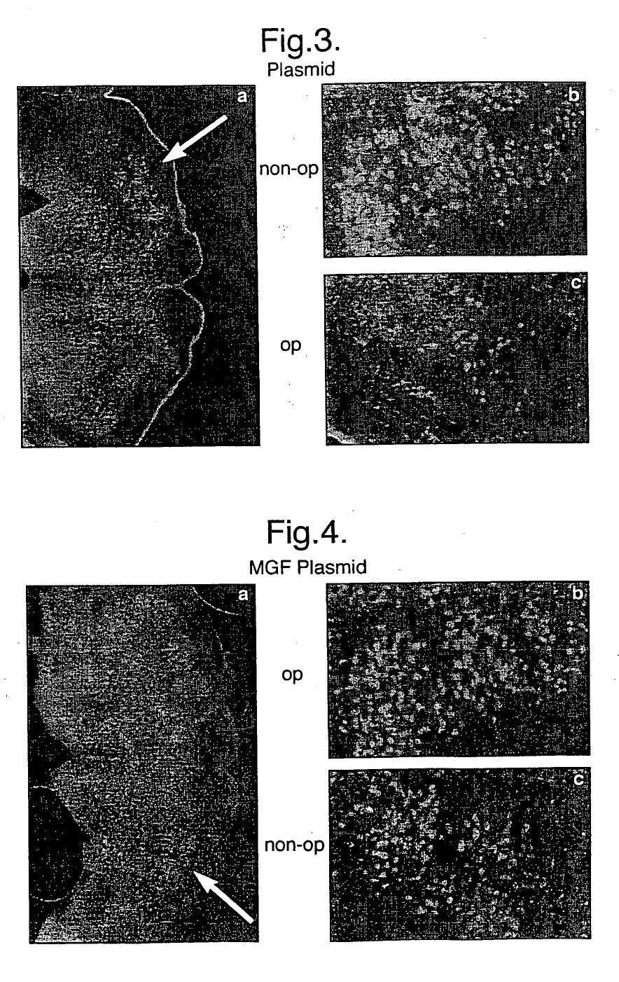

The normal adult rat facial nucleus contains approximately 3,500 motoneuroes (Table 1, FIG. 1). 1 month following nerve crush, approximately 15% of the motoneurones are lost ipsilaterally (p<0.05, Mann Whitney U test), while 1 month following nerve avulsion approximately 75% of the motoneurones are lost (FIG. 2). Injection of plasmid DNA alone into the snout 7 days before avulsion had no effect on the massive motoneuroneal loss seen 1 month later (FIG. 3). However, prior intramuscular injection of the plasmid containing the gene for L.IGF-I reduced the motoneuroneal loss 1 month following avulsion to 53% and injection of the plasmid containing the MGF gene reduced motoneuroneal loss 1 month following avulsion to 21% (FIG. 4).

TABLE 1
Total numbers of motoneurones in the facial motor nucleus 1
month following nerve avulsion (a simple tug to damage the
nerve) with or without prior intramuscular gene transfer

| | No avulsion | | Crush | | Avulsion | |
|---|---|---|---|---|---|---|
| | right | left | right | left | right | left |
| rat 1 | 3676 | 3404 | 3014 | 3619 | 884 | 3323 |
| rat 2 | 3622 | 3118 | 2889 | 3404 | 889 | 3372 |
| rat 3 | 3631 | 3385 | 2903 | 3314 | 719 | 3397 |
| rat 4 | 3666 | 3233 | 3083 | 3523 | 733 | 3023 |
| mean | 3648.7 | 3285 | 2972.3 | 3465 | 806.3 | 3278.8 |
| sd | 22.8 | 116.9 | 80.2 | 115.8 | 80.4 | 150.0 |

| | Control plasmid- | | | | | |
|---|---|---|---|---|---|---|
| | avulsion | | IGF-avulsion | | MGF-avulsion | |
| | right | left | right | left | right | left |
| rat 1 | 750 | 3384 | 1699 | 3386 | 2674 | 3624 |
| rat 2 | 798 | 3488 | 1556 | 3413 | 2934 | 3582 |
| rat 3 | 819 | 3631 | 1660 | 3438 | 2800 | 3561 |
| rat 4 | 869 | 3606 | 1640 | 3655 | 2823 | 3429 |
| mean | 809 | 3527.3 | 1638.8 | 3473 | 2807.8 | 3549 |
| sd | 42.7 | 98.8 | 52.3 | 106.7 | 92.4 | 72.9 |

Discussion

Here, we show that intramuscular gene transfer provides a means of influencing nerve/muscle interaction with the system intact. Given that fasciculation persists and motor units continue to enlarge with motoneurone diseased MND (Eisen et al, 1998), it is likely that the processes leading to motoneuroneal death continue for the most part while neuromuscular contact is maintained. The present results indicate that increased neurotrophic support from muscle prior to nerve injury can afford a long-lasting (1 month) protection against motoneuroneal death. This contrasts with the temporary protection from axotomy-induced motoneuroneal death afforded by neurotrophic factors applied at the time of injury to neonates (Vejsada et al, 1995 and 1998) and may in part reflect a method of trophic factor delivery which is better suited for molecules which need to exert local effects, which have short half lives and which are capable of inducing undesirable systemic effects (e.g. disturbances of glucose homeostasis).

We report that both IGF-I isoforms are effective in promoting adult motoneurone rescue, with the isoform produced by active muscle (MGF) being most effective. Our results implicating IGF-I in adult motoneurone rescue may also provide clues about the mechanism of motoneurone loss associated with ageing which has been documented for certain populations of cranial and spinal motoneurones since IGF-I levels decrease with ageing and mutations of a highly conserved homologue of the IGF-I receptor in Caenorhabditis Elegans has a profound effect on the ageing process and cell death. It may be the case that decreased neurotrophic support in the form of specific IGF-I isoforms, and especially MGF, increase the likelihood of motoneuroneal death either as part of the ageing process or as a result of minor trauma.

### REFERENCES

Ma\check{n}es et al, Endocrinology 138: 905-915 (1997)

Goldspink et al, J. Physiol. 4968, 1628 (1996)

McKoy et al, J. Physiol. 516.2, 583-582 (1999)

Chew et al, Endocrinology 136, No. 5 (1995)

Layall, “Transcriptional regulation of the ovine IGF-I gene”, PhD Thesis, University of Cambridge (1996)

Tobin et al, Mol. Endocrinology 1914-20 (1990)

Jansen et al, Mol. Cell Endocrinology 78: 115-25 (1991)

Rotwein et al, J. Biol. Chem. 261:4828-3 (1986)

Sharli et al, J. Physiol. 509.8, 192.8 (1998)

Yang et al, Journal of muscle cell research and cell motility 4: 487-496 (1996)

Johnson et al, Neuroscience 84: 141-150 (1998)

Eisen et al, “Amyotrophic Lateral Sclerosis” (Cambridge University Press, Cambridge, 1998)

Vejsada et al, Eur. J. Neurosci. 7: 108-115 (1995)

Vesjada et al, Neuroscience 84: 129-139 (1998)

1-18. (canceled)

19. A method of treating a neurological disorder comprising administering to a subject in need thereof a composition comprising an effective amount of an MGF (mechano-growth factor) Insulin-like Growth Factor I (IGF-I) isoform comprising amino acid sequences encoded by nucleic acid sequences of IGF-I exons 4, 5 and 6 in the reading frame of MGF and having the ability to reduce motoneurone loss by 20% or greater in response to nerve avulsion.

20. A method according to claim 19 wherein the MGF has the ability to reduce motoneurone loss by 50% or greater or 80% or greater in response to nerve avulsion.

21. A method according to claim 19 wherein the MGF is unglycosylated.

22. A method according to claim 19 wherein the MGF has:
(a) the sequence of Human MGF (SEQ ID NO. 2), Rat MGF (SEQ ID NO. 4) or Rabbit MGF (SEQ ID NO. 6);
(b) a sequence having 70% or greater homology to a sequence of (a);
(c) a sequence comprising the amino acids encoded wholly or partly by exons 4, 5 and 6 of human, rat or rabbit MGF DNA of SEQ ID NO. 1, 3 or 5, or a sequence having 70% or greater homology thereto; or
(d) a sequence encoded by a nucleic acid sequence capable of selectively hybridising to a sequence of (a), (b) or (c).

23. A method manufacturing a medicament for the treatment of a neurological disorder comprising expressing a polynucleotide encoding an MGF (mechano-growth factor) Insulin-like Growth Factor I (IGF-I) isoform comprising amino acid sequences encoded by nucleic acid sequences of IGF-I exons 4, 5 and 6 in the reading frame of MGF and having the ability to reduce motoneurone loss by 20% or greater in response to nerve avulsion.

24. A method according to claim 23 wherein the polynucleotide comprises the coding sequence of SEQ ID NO. 1, 3 or 5.

25. A method according to claim 24 wherein the polynucleotide is contained within a vector.

26. A method according to claim 25 wherein the vector is a plasmid vector or a disarmed viral vector.

27. A method according to claim 19 wherein the neurological disorder is a disorder of motoneurones and/or a neurodegenerative disorder.

28. A method according to claim 27 wherein the effects of the treatment comprise motoneurone rescue.

29. A method according to claim 28 wherein the effects of the treatment comprise adult motoneurone rescue.

30. A method according to claim 27 wherein the disorder is selected from amyotrophic lateral sclerosis; spinal muscular atrophy; progressive spinal muscular atrophy; infantile or juvenile muscular atrophy, poliomyelitis or post-polio syndrome; a disorder caused by exposure to a toxin, motoneurone trauma, a motoneurone lesion or nerve damage; an injury that affects motoneurones; motoneurone loss associated with ageing; autosomal or sex-linked muscular dystrophy; diabetic neuropathy; and peripheral neuropathies.

31. A method according to claim 19 wherein the composition further comprises another neurologically active agent or wherein treatment with the MGF is carried out in combination with another neurologically active agent.

32. A product comprising an MGF IGF-I isoform comprising amino acid sequences encoded by nucleic acid sequences of IGF-I exons 4, 5 and 6 in the reading frame of MGF and having the ability to reduce motoneurone loss by 20% or greater in response to nerve avulsion or an MGF-encoding polynucleotide encoding said sequences and another neurologically active agent for simultaneous, separate or sequential use in the treatment of a neurological disorder.

33. A product according to claim 32 for use in the treatment of a disorder of motoneurones and/or a neurodegenerative disorder.

34. A pharmaceutical composition comprising an MGF IGF-I isoform comprising amino acid sequences encoded by nucleic acid sequences of IGF-I exons 4, 5 and 6 in the reading frame of MGF and having the ability to reduce motoneurone loss by 20% or greater in response to nerve avulsion or an MGF-encoding polynucleotide encoding the same, another neurologically active agent and a pharmaceutically acceptable carrier.

35. A method of treating a neurological disorder comprising administering to a subject in need thereof an effective amount of an IGF-I isoform as defined in claim 34.

36. A method according to claim 31, wherein the other neurologically active agent is a polypeptide growth factor or a nucleic acid encoding a polypeptide growth factor.

37. A composition according to claim 34, wherein the other neurologically active agent is a polypeptide growth factor or a nucleic acid encoding a polypeptide growth factor.
